(19) United States
(12) Patent Application Publication
(10) Pub. No.: US 2021/0275713 A1
(43) Pub. Date: Sep. 9, 2021

(54) PERSONAL AIR PURIFICATION MASK STERILIZING INSERT

(71) Applicant: Robin J. MCDANIEL, Two Rivers, WI (US)

(72) Inventor: Robin J. MCDANIEL, Two Rivers, WI (US)

(21) Appl. No.: 16/811,134

(22) Filed: Mar. 6, 2020

(51) Int. Cl. A61L 9/20; A41D 13/11

(52) U.S. Cl. CPC A61L 9/20; A61L 2209/134; A61L 2209/12; A41D 13/11

(57) ABSTRACT

An improved invention relating to protective breathing devices in general, and more specifically describes an apparatus to economically and effectively mitigate against airborne viral infection. Used as an insert in conjunction with a variety of other masks, powered air purifying respirators (PAPR), and breathing devices; this device combines an internal air deflection shell and with a miniaturized high-intensity shortwave UV light source to sterilize airborne germs. Can also be used with filtering facepiece respirators (FFRs) as a between use disinfecting system for reuse.

The personal air purification mask sterilizing insert serves as an air mask infection mitigating insert while donned and a disinfection device while doffed. Facilitating the conservation of precious resources in that the design is more elegantly simple affording lower cost and ease of operation while providing for reuse of N95 FFRs.

This way, disposable N95 masks can be recycled and used more than once to protect people against infection, and conserve medical supplies in view of shortages in supplies in outbreak situations.

### FEDERALLY SPONSORED RESEARCH

Not Applicable

### Current U.S. Class: 128/201.245, 128/201.28; 128/201.29

### Current International Class: A62B 18/00

### Field of Search: 128/201.12-202.11

### BACKGROUND AND FIELD OF THE INVENTION

This invention relates to protective breathing devices in general, and more specifically describes an apparatus for economically and effectively mitigating against airborne viral infection. Used as an insert in conjunction with a variety of other masks and breathing devices this device combines an internal air deflection shell and with a miniaturized high-intensity shortwave UV light filtering facepiece respirator (FFRs) disinfecting system. This invention also applies to powered air purifying respirators (PAPR).

### BACKGROUND DESCRIPTION OF PRIOR ART

### References

U.S. PATENT DOCUMENTS

| | | | |
|---|---|---|---|
| 6,797,044 | 2004 | Chieh Ou Yang | 96/244 |
| 5,957,131 | 1999 | Hutchinson | 128/206.17 |
| 10,485,946 | Nov. 26, 2019 | Reese | 128/200.27 |
| 8,397,715 | Mar. 19, 2013 | Litz | 128/201.25 |

Pure clean air is a basic requirement for all life on the surface of the Earth.

Masks have been developed by people over the centuries in order to clean the air prior to breathing said air. Historical accounts recorded the use of “Plague Masks” in medieval times to protect the health care workers of that time from unseen agents of sickness. There exists a large set of examples of prior art with regards to gas masks and gas breathing respirators. Most conventional gas mask/respirators utilize a partial or full mask that covers the nose and mouth and in some cases the eyes and entire upper face.

Conventional gas mask/respirators invariably utilized a removable cartridge containing various filter media or in their simplest form the cartridges incorporate a filter cloth of charcoal or multi-layered polymer material that merely filters out particulate materials including aerosol and colloidal suspensions. Those filters that rely on activated charcoal eliminate particulate material and absorb vapor and gas contaminants as they enter and are absorbed by the activated charcoal. Most effective filtration media utilizes a HEPA type filter formed of pleated filter paper with minute spaces for allowing the passage of air through the mask.

Despite their effectiveness against particulate materials in suspensions, the foregoing and other known filtering gas masks are not sufficiently effective in filtering biological organisms such as viruses, bacteria, and other germs. With the increased threat of biological warfare, accidental release of contagion, disease and other airborne pathogens, there is currently a significant need for gas mask/respirators capable of killing or mitigating against germs as well as filtering toxins.

There would be a great advantage in a breathing gas mask, respirator, or even simple a face mask that included a high-intensity shortwave UV light disinfecting system, which would be easy to insert thus compatible with existing masks or respirator systems.

Prior approaches and some previous inventions include a room sized AIR FILTER DEVICE, (Yang, 2004) Including air filter net and one or more ultraviolet light emitting-diodes (UV-LEDs). The UV LED is disposed on the exhaust device located at one side of the filter net to irradiate UV light toward the filter net accomplishing filtering and sterilizing effects to the air passing through the exhaust device and thus improving the quality of indoor air.

A BIOLOGICAL WARFARE MASK, (Hutchinson et. al., 1999) A filtration mask that offers protection from hazardous particulate aerosols, vapors, and the like and which is particularly useful for providing respiratory protection from biological warfare agents. The mask provides a military level of protection against biological agents in a half mask configuration. Yet this invention did not provide for UV light disinfection of contaminants which passed thru the filters.

A PERSONAL HEALTH DEVICE, (Reese, 2019) a Portable device provided filtered air to the face of a user and a UV light source provided inside the housing providing UV light to purify air moving inside said housing toward the filter member. This device was portable in nature as it could be placed on a table and even included a pill box and medications log.

A CHEMICAL AND BIOLOGICAL PROTECTION MASK (Litz, Burnie, 2013) a mask system that combines a filter cartridge with a miniaturized high-intensity shortwave UV disinfecting system in a compact mask system. This device utilized a complex set of UV illumination tube attachable between the inhalation valve coupling and filtered canister. The UV illumination tube further comprises an aluminum tube, a plastic insert positioned inside the aluminum tube, and a plurality of circuit boards seated within the insert. The circuit boards each have a plurality of UV LED lights axially mounted thereon to irradiate and disinfect air passing through the UV-illumination tube powered by a battery connected to a voltage regulator, a current chopping circuit for providing pulsed current thereto. Included is an optional solar cell for powering the device.

The current state-of-the-art comprises a variety of devices some more complex than others, where the concept of UV sterilization has been incorporated. These devices are combinations of larger HVAC, table-top devices, masks, or respirators. Most of these devices are complex assemblies of multiple technologies and are therefore more expensive than simple hospital face masks or surgical masks.

The possibility of a pandemic or national outbreak of a contagion necessitates the distribution and use of simpler face masks. Many of the simple face masks or filtering facepiece respirators (FFRs) such as the N95 rated variety are designed to be single use disposable and yet few are reusable in design. During a pandemic of influenza or other viral airborne infections the demand for such face masks will be very high, and supplies may be limited.

The problem and possible solution to shortages of FFRs may be found in decontamination and reuse (FFR-DR). In a major article Ultraviolet germicidal irradiation of influenza-contaminated N95 filtering facepiece respirators, (D. Mills et al./American Journal of Infection Control 46 (2018) e49-e55) research is published.

Other high-tech respirators and mask systems will also need protection from leakage or bypass of virus contagion.

### SUMMARY OF THE INVENTION

The present invention is specified in view of the aforesaid problems in the related art.

The personal air purification mask sterilizing insert serves as an air mask infection mitigating insert while donned and a disinfection device while doffed. Facilitating the conservation of precious resources in that the design is more elegantly simple affording lower cost and ease of operation while providing for reuse of N95 FFRs.

It is, therefore, an object of the present invention to provide a personal air purification mask sterilizing insert.

It is another object to provide a portable personal air purification mask sterilizing insert.

It is another object to provide a lightweight personal air purification mask sterilizing insert with a miniaturized high-efficiency UV LED-light source.

It is another object to provide a personal air purification mask sterilizing insert with an internal air-flow shell and diffuser member.

It is another object to provide a personal air purification mask sterilizing insert with a cover to facilitate the doffed mode disinfecting operation.

These and other objects are achieved herein by a personal air purification mask sterilizing insert system that combines the internal air-flow shell with a miniaturized high-efficiency UV LED-light source, and a diffuser member. The assembly of objects having the UV LED-light source positioned between the air-flow shell and the diffuser member. The UV LED-light source preferably emits light within a range of from 275 nm˜285 nm wavelength which disinfects the air flowing within the assembly and the filter elements exposed to said UV light in proximity to the mask sterilizing insert.

### BRIEF DESCRIPTION OF THE DRAWINGS

Illustrative and presently preferred embodiments of the invention are shown in the accompanying drawings in which:

FIG. 1 “Invention while donned”, is a side view of a representation showing the components of a personal air purification mask sterilizing insert, according to one embodiment of the present invention while used as an insert to any variety of mask/respirators;

FIG. 2 “Invention while doffed”, is a side view of a representation showing the components of a personal air purification mask sterilizing insert, according to one embodiment of the present invention while used to disinfect an N95 filtering facepiece respirator (FFR-DR) prior to reuse.

### REFERENCE NUMERALS USED IN DRAWINGS

1 Air-flow shell
2 Air passage convolutions
3 UV LED-light source
4 Power conduit
5 Power controller
6 Diffuser
7 Air passage holes
8 Disinfecting cover
9 Filter facepiece respirator

However, before proceeding with the description, it should be noted that the various embodiments shown and described herein are exemplary only and are not intended to represent the extent to which the present invention may be utilized. Indeed, the systems and methods described herein could be readily applied to any of a wide range of personal air purification mask sterilizing insert designs, as would be obvious to persons having ordinary skill in the art after having become familiar with the teachings provided herein. Consequently, the present invention should not be regarded as limited to the described personal air purification mask sterilizing insert system and example configurations shown and described herein.

### DETAILED DESCRIPTION

The present invention is a personal air purification mask sterilizing insert system.

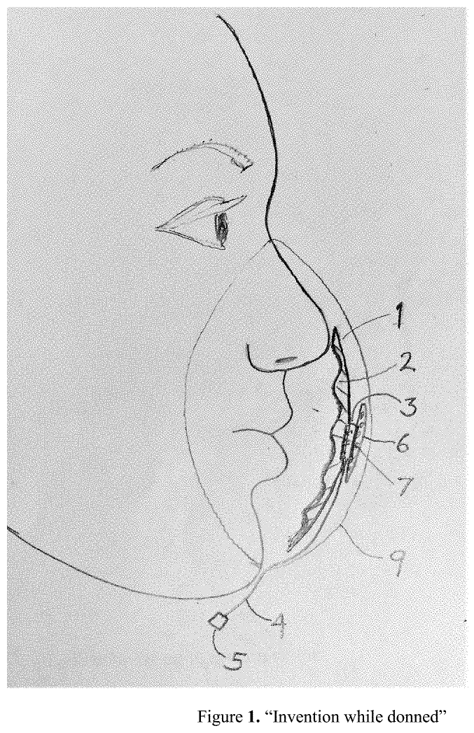

Referring now to FIG. 1: “Side view of Invention while donned”, one embodiment of a personal air purification mask sterilizing insert may comprise an Air-flow shell 1 formed with Air passage convolutions 2 to provide a plurality of air passage convolutions leading to a plurality of UV LED-light source 3 (powered by electricity flowing thru a Power conduit 4 from a portable battery pack attached to a Power controller 5), which then radiates UV light within a range of from 275 nm˜285 nm wavelength which disinfects the air flowing within the assembly, and the filter elements exposed to said UV light in proximity to the mask sterilizing insert. When the mask/respirator user inhales, and sufficient air-flow is achieved, the air flows generally thru the mask/respirator filter(s) and a portion of air flows past the UV LED-light source 3, a portion of the air flows past the Diffuser 6, a portion of the UV Light and air flows thru the Diffuser via Air passage holes 7, via a created difference in pressure, irradiating potential viral or bacteriological pathogens to mitigate infection to the user. And also both conversely upon exhale and sufficient air-flow is achieved, a portion of exhaled air flows generally past the UV LED-light source 3 a portion of the air flows past the Diffuser 6, a portion of the UV Light and air flows thru the Diffuser via Air passage holes 7, via a created difference in pressure, then thru the mask/respirator filter(s) and irradiating potential pathogens to mitigate infection from the potentially infected user.

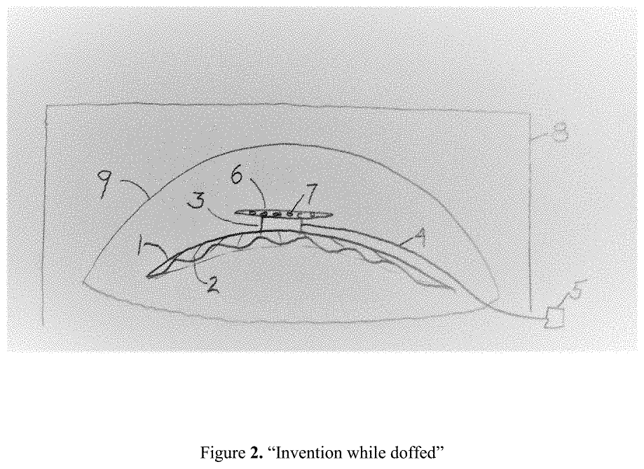

Referring now to FIG. 2: “Side view of Invention while doffed”, one embodiment of a personal air purification mask sterilizing insert may comprise an Air-flow shell 1 attached to a plurality of UV LED-light source 3 (powered by electricity flowing thru a Power conduit 4 optionally from a stationary power source or battery attached to a Power controller 5), which then radiates UV light within a range of from 275 nm˜285 nm wavelength, a portion of the UV light shines past the Diffuser 6, a portion of the UV Light shines thru the Diffuser 6 via Air passage holes 7, which disinfects the exposed filter elements from light shining out from the assembly, and the filter elements exposed to said UV light in proximity to the mask sterilizing insert. The previously used N95 filtering facepiece respirator 9 (FFR-DR) may be optionally positioned over a personal air purification mask sterilizing insert (both placed on a suitable surface) and a Disinfecting cover 8 with a reflective interior surface is placed over both while the system is powered on allowing UV light to shine within the cover, in one envisioned embodiment the cover may be lined with a reflective material.

In summation, then, because persons having ordinary skill in the art could readily select from one or several component configurations of the design described herein, after having become familiar with the teachings of the present invention, the present invention should not be regarded as limited to varying any one or combination of personal air purification mask sterilizing insert components described herein.

Present invention should not be regarded as limited to use with any specific mask/respirator.

Present invention should not be regarded as limited to use with any specific filtering facepiece respirator.

Present invention should not be regarded as limited to any kind of air breathing animal.

Present invention should not be regarded as limited to any scale of air flow.

Present invention should not be regarded as limited to any source of air/gas flow Present invention should not be regarded as limited to any kind of breathing gas.

Present invention should not be regarded as limited to any kind of power source or controller.

Present invention should not be regarded as limited to any kind of cover or box serving as Disinfecting cover.

Present invention should not be regarded as limited to any use of materials for components, Air-flow shell, Diffuser, or Power controller.

Having herein set forth preferred embodiments of the present invention, it is anticipated that suitable modifications can be made thereto which will nonetheless remain within the scope of the invention. The invention shall therefore only be construed in accordance with the specific included claims.

I claim the invention is:

1. A personal air purification mask sterilizing insert for use while donning a respirator, comprising:
an internal air-flow shell for directing the flow of air;
a UV LED-light source attached disposed to the air-flow shell, said UV LED-light source being a plurality of UV LEDs disposed to the air-flow shell;
a perforated diffuser in proximity to the UV LED-light source such that the UV LED-light source is located between said diffuser and said air-flow shell;
a power controller connected to the UV LED-light source.

2. A personal air purification mask sterilizing insert according to claim 1, wherein all of said plurality of UV LEDs emit light within a range of from 275 nm to about 285 nm wavelength.

3. A personal air purification mask sterilizing insert for use while doffing a mask/respirator, according to claim 2, further comprising a reflective cover wherein a previously used, potentially contaminated, N95 filtering facepiece respirator is place over the personal air purification mask sterilizing insert then both are covered while the insert is powered on during a disinfection cycle.
